(19) United States
(12) Patent Application Publication
(10) Pub. No.: US 2025/0178189 A1
(43) Pub. Date: Jun. 5, 2025

(54) ELONGATED FUNCTIONAL SYSTEM CONFIGURED TO BE ADVANCED IN THE LUMEN OF A PIPE, A DUCT OR A TUBE

(71) Applicant: BASECAMP VASCULAR, Reims (FR)

(72) Inventors: Jean-Baptiste CAZENEUVE, Ivry-sur-seine (FR); Camille MAIANO, Brélès (FR)

(73) Assignee: BASECAMP VASCULAR, Reims (FR)

(21) Appl. No.: 19/047,808

(22) Filed: Feb. 7, 2025

Related U.S. Application Data
(63) Continuation-in-part of application No. 17/044,962, filed on Oct. 2, 2020.

(30) Foreign Application Priority Data
Apr. 12, 2018 (EP) 18305452.7

(51) Int. Cl. B25J 9/06; A61M 25/01

(52) U.S. Cl. CPC B25J 9/065; A61M 25/0158; A61M 2025/0161; A61M 2205/0266; A61M 2205/0294

(57) ABSTRACT

An elongated functional system configured to be advanced in the lumen of a pipe, a duct or a tube. The system has a body part having a main proximal part and a distal part, the distal part being a continuous extension of the proximal part, the distal part including one functional end terminating with a tip, and at least one active area located upstream the functional end on the distal part; and an actuator configured to transform an amount of energy to the distal part sufficient to cause a reversible and multiple curvature of the active area, thereby preventing undesired spring back of the whole system; the actuator being connectable to a source of energy.

### FIELD

The present invention relates to devices for use for insertion in a pipe, a duct or a tube. According to the invention, the device is to be operated from outside of the pipe, duct or tube. In one embodiment, the device of the invention is a medical device, especially an invasive device for introducing, guiding, advancing, emplacing or/and holding medical devices, such as for example, catheters or endoscopes, in veins or arteries.

### BACKGROUND

There is a wide range of applications where there is a need for using a system inside a pipe, a duct or a tube, for example for the placement of the distal part of a tube in a specific location, for bringing light or chemicals, or for bringing a functionality at a remote or hard-to-access place.

When advancing a system in the lumen of a pipe, duct or a tube, it is important that the user can control carefully and precisely the movement and the placement of the system. Placement of systems within pipes is a known technical issue in oil engineering or in motor engineering. Placement of systems within body tubes, such as for example the ostium, is also known as being a challenge in the medical field.

For example, EP 2 687 254, describes a coronary artery catheter adapted for introducing its distal part into a coronary ostium through an artery. In this document, the catheter comprises, in its natural state, several portions of different curvatures. One drawback of this system is the difficulty of introducing the catheter within the ostium and the need of using a catheter introducer. Another drawback of this system is that, when attending to enter the ostium, a snapping effect (i.e. sudden detorsion of the catheter, which has undergone torsion during introduction) may occur.

The patent document published under the number U.S. Pat. No. 5,322,509 provides a cardiac catheter which may avoid the need for catheter exchange to access both ostia. Instead of a C-shaped distal part, the catheter described in U.S. Pat. No. 5,322,509 includes a distal portion attached to the intermediate section and consists of a double or a reverse curve (S-shaped). The structure allows the catheter to be utilized to enter either left or right ostium without catheter exchange by rotation of the catheter about its axis. However, this system comprises a plurality of curves. Therefore, during the progression of said guiding means in the aortic artery, the device contacts and pressures the walls of the artery, and can be harmful for the patient.

The patent document published under the number CA 2105774 discloses a catheter particularly adapted for cardiac ablations, comprising an electrode tip assembly that is bendable at the selection of the user in two different directions. The electrode tip assembly assumes a different predetermined curve configuration when bent in the two directions. However, the curvature movements are limited. Indeed, only one curvature may be controlled by a surgeon. Moreover, this catheter cannot be inserted in a complex duct.

The Applicant carefully observed the snapping phenomena in various situations and could make sure that, systematically, when a system's most distal part is pushed forward in a curve, it may touch an edge then a sudden and undesired spring back (snapping) of the whole system may occur, making the system uncontrolled and difficult to reposition. Such undesired rotation is typically the result of a spring-back effect due to prior system torsion followed by a partial or full elastic return when the applied constraint is released.

This invention aims at proposing an elongated system capable to stabilize its distal part in the direction wanted by the user (whatever the curvature of its environment), and capable to avoid or hinder any spontaneous undesired torsional spring-back effect of the whole system.

### SUMMARY

The present invention relates to an elongated functional system for implementing the anti-snapping method described above, the system being configured to be advanced in the lumen of a pipe, a duct or a tube, said system having:
(i) a body part having a main elongated proximal part and a distal part, said distal part being a continuous extension of the proximal part, said distal part comprising one functional end terminating with a tip, and at least one active area located upstream the functional end on the distal part; and
(ii) an actuator configured to convey or transform an amount of energy to the distal part sufficient to cause a reversible multiple curvature of the active area, one curvature among the multiple curvature being greater or equal to the curvature of the center of the tube, this tube curvature being supposed to be non zero, at the location of the active area, thereby preventing undesired spring back of the whole system; the actuator being connectable to a source of energy.

In one embodiment, the actuator and, thereby, its corresponding active area, are distant from the tip, the distance between the distal end of the tip and the distal end of the actuator ranging from 0.1 to 5 times the length of the actuator; in one embodiment, the distance between the distal end of the tip and the distal end of the actuator preferably ranges from 0.5 to 3 or 0.1 to 2 times the length of the actuator.

In one embodiment, the body part is a solid body part. In one embodiment, the body part does not comprise any lumen.

The activation of the first actuator stabilizes the elongated system in case an edge is touched during introduction in the pipe, duct or lumen. At this stage, the distal end might touch such edge and trigger an undesired torsional spring-back. By actuating the first actuator, such phenomenon is controlled.

In one embodiment, the elongated functional system further comprises at least one second actuator closer to the tip than the first actuator described above, configured to activate the functional end. In one embodiment, the at least one first and at least one second actuators are not connected or coupled one to another. In one embodiment, there is no electrical continuity between two actuators. In one embodiment, the actuators do not form a zigzag pattern. In one embodiment, the system does not comprise any electrode.

In one embodiment, the length of the actuators ranges from 1 to 100 mm, preferably 5 to 50 mm. In one embodiment, the length of the actuator of the proximal active area ranges from 1 to 100 mm, preferably 5 to 50 mm. In one embodiment, the length of the actuator of the functional end, when present, ranges from 1 to 100 mm, preferably 5 to 50 mm.

In one embodiment, the distance between the distal end of the tip and the distal end of the actuator of the proximal active area ranges from 20 to 300 mm. In one embodiment, this distance ranges from 20 to 100 mm. In another embodiment, this distance ranges from 50 to 300 mm.

In one embodiment, the distance between the distal end of the tip and the distal end of the actuator of the proximal area ranges from 0.5 to 3 times the length of the actuator of the proximal active area.

In one embodiment, the main elongated proximal part and/or the distal part are straight. In one embodiment, the main elongated proximal part and/or the distal part are straight and flexible. In one embodiment, the distal part is hook-shaped. In one embodiment, the distal part is hook-shaped and flexible.

In one embodiment, the functional end is flexible. In one embodiment, the functional end is either straight or curved by design, which means that when free, it naturally forms a curve, for example is hook-shaped.

In one embodiment, the at least one actuator is mechanically fixed or anchored to whole or part of the body part, so that the activation of the at least one actuator generates a curvature in at least one location of the body part. In one embodiment, the at least one actuator is configured to convey or transform an amount of energy to the distal part of the body part sufficient to cause a reversible curvature of the active area, corresponding to the curvature of the tube at the location of the active area; in one embodiment said curvature is greater or corresponds (which means is equal to), in angle and/or in radius to the curvature of the center of the tube (where the system is inserted) at the location of the active area. The reversibility is obtained once the actuator does not act anymore on the active area (which generally means that it is switched off). In one embodiment said curvature is greater than the curvature of the center of the tube (where the system is inserted) at the location of the active area (which means that the radius of curvature of the active area is less than that of the tube). In one embodiment, the actuators are mechanically fixed to whole or part of the distal part of the system, eventually at different locations on the body part of the system.

In one embodiment, the actuators are held against the body part continuously or discontinuously. In one embodiment, the actuators are held against the body part by any means or restraint device effective for the actuator to transmit a movement to the body part.

In one embodiment, at least one of the actuators is longitudinal and extends along the distal part of the system. In one embodiment, the actuator(s) contract(s) and/or curvate(s) when stimulated by an energy source, thereby: causing the flexion of the area to which they are fixed (thereby called active area), and/or activating the functional end. In one embodiment, the active area is activated by an actuator and exhibit a curvature, and the functional end is not activated and is straight or curved by design. In one embodiment, the active area is activated by an actuator and exhibit a curvature, and the functional end is activated and exhibit a curvature in a direction or a plan identical or different than the direction or plan of the active area. According to one embodiment, the at least one actuator is fixed to the distal part of the body part and exhibit a predefined contraction (shortening of its length) and/or a curvature, or a translation towards the proximal end, when actuated by means of an energy source. Said energy source may be electric current, hydraulic fluid pressure, thermal energy, magnetic energy or mechanical energy.

In one embodiment, the elongated functional system according to the invention comprises at least one first actuator configured to cause a curvature of said active area. In one embodiment, the elongated functional system according to the invention comprises a only one actuator (called first actuator above) configured to cause a curvature of said active area. In one embodiment, the elongated functional system according to the invention comprises at least one first actuator configured to cause a curvature of said active area and at least one second actuator configured to activate the functional end. In one embodiment, the elongated functional system according to the invention comprises a single first actuator configured to cause a curvature of said active area and a single second actuator configured to activate the functional end.

Activating the functional end can be, for example, controlling the orientation of the functional end and/or triggering a functionality of the functional end, such as for example switching on a light arranged on the tip of the system or releasing the content of a compartment located in the functional end. In one embodiment, the functional end is a steerable end.

In one embodiment, the actuators are lightweight and capable of large deformations. In one embodiment, actuators, or each independently first actuator and second actuator are made of, or comprise, at least one shape memory alloy and/or of at least one polymer and/or at least one metal and/or at least one piezo electric material. In one embodiment, the actuator, or each independently first actuator and second actuator and the body part are each independently made of shape memory alloy, preferably of nickel-titane alloy, commonly referred to as nitinol. In one embodiment, the body part is an optical fiber. According to one embodiment, said material may change its dimension or its shape when energized, stimulated or activated. In this invention, it is made clear that the words energized, stimulated and activated can be used one for another and mean that an energy from the source of energy is transmitted, by any suitable means, to the actuator.

In one embodiment, the first actuator and/or the second actuator, each independently, contracts or curvates when stimulated by a source of energy, thereby respectively: causing the flexion or curvature of the active area to which they are fixed, and/or activating the functional end.

In one embodiment, the material is a shape memory alloy which has been elongated before use (compared to its original shape) and returns to its original shape when activated. In one embodiment, the actuator is at least one wire. In one embodiment, the actuators are shape memory alloy wires. In one embodiment, the actuator is at least one cable. In one embodiment, the actuator is made of polymer.

In one embodiment, actuation of the single actuator generates a double or a multiple curvature of active area and/or functional end. In one embodiment, the actuator and/or the first actuator and/or the second actuator, when activated, trigger a simple or a double or a multiple curvature of the distal part of the system, on whole or part of the length of said distal part. In one embodiment, the simple or double or multiple curvature is selected in the group consisting of a S-shape, a C-shape, L-shape, J-shape, a U-shape or a G-shape of whole or part of distal part.

In one embodiment, the elongated functional system of the invention has:
(i) a body part having a main proximal part and a distal part, said distal part being a continuous extension of the proximal part, said distal part comprising one functional end terminating with a tip, and at least one active area located upstream the functional end on the distal part; and
(ii) at least two actuators configured to convey or transform an amount of energy to the distal part sufficient to cause a reversible curvature of the active area, thereby preventing undesired spring back of the whole system;
(iii) the two actuators being arranged on the outside of the body part; or within the wall of the body part, with an angular shift of 0 to 180°, preferably more than 0° to 180°; more preferably 90 to 180°;
(iv) the actuator being connectable to a source of energy.

In one embodiment, the angular shift of the actuators is such that the active area and the functional end are, upon activation, in secant plans or exhibit different or opposite direction of curvatures. In one embodiment, the actuators may trigger different radius of curvature for each one of the active area and the functional end.

In one embodiment, the elongated functional system according to the invention further comprises an external control unit located at the proximal end of the proximal part, said control unit comprising at least one controller device configured to actuate each actuator independently.

In one embodiment, the elongated functional system according to the invention includes a first and a second actuator and the first actuator and the second actuator may be spaced one from another or may be arranged one after the other.

In one embodiment, the active area comprises the at least one third actuator.

According to one embodiment, the elongated functional system of the invention further comprises a source of energy and means for providing energy to the actuator connected to said source of energy. According to one embodiment, said electric energy source is a battery, a generator or an accumulator. According to one embodiment, the elongated functional system of the invention further comprises means to provide/transmit energy to the first and/or said second actuator in a controlled manner. According to one embodiment, said means to provide/transmit energy to said first and/or said second actuator are at least one conductive wire.

In one embodiment, said elongated functional system further comprises a handle positioned at the proximal end of the main body proximal part. In one embodiment, said handle comprises an at least one first controller device configured to actuate independently the actuator(s) of the active area and an at least one second controller device configured to actuate independently the actuator(s) of the functional end. The handle advantageously may help actuating the system.

In one embodiment said elongated functional system is an endoscope, a catheter or catheter guide, preferably a catheter guide.

In one embodiment, the two actuators are aligned one after the other longitudinally along the longitudinal axis of the elongated system. This allows a better displacement within any duct or pipe or lumen.

In a second aspect, this invention relates to a method for stabilizing an elongated functional system advanced in the lumen of a tube having at least one curve, wherein the elongated functional system has:
a body part having a main proximal part and a distal part, said distal part being a continuous extension of the proximal part, said distal part comprising one functional end terminating with a tip, and at least one active area located upstream the functional end on the distal part; and
an actuator configured to convey or transform an amount of energy to the distal part sufficient to cause a reversible curvature of the active area, greater or equal to the curvature of the center of the tube, at the location of the active area, thereby preventing undesired system spring back; the actuator being connectable to a source of energy,
the method comprising the steps of:
advancing the system in a curve of the tube;
stabilizing the system by actuating the active area so that it reaches a double curvature, one curvature of the double curvature being equal or greater than that of the curve of the tube.

The present invention also relates to a catheter comprising the elongated system according to the present invention. This invention thus provides an elongated functional system for guiding an elongated system (which can be, for example but not limitatively, a catheter), from a predefined trunk duct (for example the aortic arch) to a cross duct extending from the trunk duct (for example an artery).

The present invention also relates to the use of the elongated functional system in a method comprising the steps of:
advancing the system in order to align the first curve of the proximal active area 3p of the distal part with the curve of the tube where the system is advanced, for example the aortic arch;
actuating the active area 3p;
positioning the distal part of the elongated functional system downstream the targeted tube, for example the targeted artery;
optionally, actuating the distal active area 3d of the distal part; and
pulling the elongated functional system in the proximal direction until introducing said second active area in the targeted tube, for example the targeted artery.

In one embodiment, the targeted artery is the brachiocephalic artery, the left common carotid artery or the left subclavian artery and the trunk artery is the aortic arch.

### Definitions

In the present invention, the following terms have the following meanings:
“functional end”: is the most distal end of the system; it can be functionalized by an actuator in order to bend, according to a plane, a direction, and according to a wished angle and radius of curvature. It can also be functionalized with a light, or a tank, or a balloon, for example for the release of pharmaceutical products. Having both an actuator and another function is an embodiment of the invention.
“actuator”: can be any type of string, cable, wire, ribbon, tube or any set of those, capable of activating the body part to which it is fixed in order to trigger a function or to induce a bending of an area of the body part to which it is fixed. Actuators are materials and devices that are able to change their shape in response to changes in environmental conditions and perform mechanical work. An actuator may convey energy. Most of the time, an actuator transforms the received energy into another type of energy. In one embodiment, the actuator receives heat, and upon reception of the heat, it contracts.
“active area” should be understood as a zone or a region of the system which is in relation with at least one actuator. In one embodiment, an active area is an area to which an actuator is fixed. In one embodiment, the active area is an area capable of curvating when at least one actuator, fixed at least at the limits of the area, is activated.
“catheter” is a tubular medical device for insertion into canals, vessels, passageways or body cavities for diagnostic or therapeutic purposes such as to permit injection/withdrawal of fluids, to keep passageways open, to inspect internal organs and tissues and to place medical tools into position for medical treatment within the body of an animal or of a human. In this invention, the term “catheter” encompasses any cannula or medical probe designed for insertion in a human or animal canal, vessel, passageway or body cavity.
“snapping” refers to a sudden and undesired torsional spring back of an elongated structure. When an elongated structure is actively bended along one of its portion it may contact and deform its surrounding environment. In this case, the environment is accumulating energy due to elastic deformation. At a certain level of accumulated energy and if the elongated structure is flexible in torsion, the elongated structure will naturally twist along its own axis. It will twist so that its bended part will reorient and apply less deformation to the environment (deformation energy is released). We say in this case that the elongated structure “snaps” to a new configuration by torsion. Unforeseen snapping is clearly not desirable and could be dangerous in surgical applications.
“wire” refers to a longitudinal means comprising a length sensibly higher than its thickness, its width or its diameter. In one embodiment the wire is a strand. In another embodiment, the wire is a very flexible rod. In one embodiment, the diameter of the wire ranges from 0.01 mm to 1 mm.
“to curvate” means to take the form of a curvature, to bend. Having a curvature or being curved is used in opposition to being straight. The term curvature means non-zero curvature. The curvature can be positive or negative. Mathematically speaking, the curvature of a circle is equal to the ratio of the angle of an arc to its length. It is the result of a flexion in the present invention.
“reversible curvature” means therefore a curvature able to go back initial state before actuation.
“about” when placed before a figure, means plus or minus 10% of the figure.

### BRIEF DESCRIPTION OF THE DRAWINGS

FIG. 1 is a drawing showing the distal part of an elongated functional system wherein the first actuator and the second actuator are actuated.

FIG. 2 is a drawing showing an elongated functional system comprising a handle, a first actuator and a second actuator.

FIG. 3 is a drawing showing an elongated functional system comprising connective wires.

FIG. 4 is a drawing showing an elongated functional system (1) introduced in the aortic arch in order to reach the supra aortic trunk ostium targeted.

FIG. 5 is a drawing showing the distal part of an elongated functional system wherein a single actuator is actuated and generates a multiple curvature.

FIG. 6 is a drawing showing a partial view of the distal part of an elongated functional system, further illustrating the fixation system between an actuator and a body part of the system.

### REFERENCES

1—Elongated functional system
2—Proximal part
3—Distal part
3p—Active area
3d—Functional end
3t—Tip
30—Actuator of distal part
30p—Actuator of active area
30d—Actuator of functional end
4—Conductive mean
4p—First conductive means
4d—Second conductive means
5p—First controller
5d—Second controller
6—body part
7—Sheath
8—Mechanical link

### DETAILED DESCRIPTION

This invention relates to an elongated system 1.

One goal of this invention is to be able to counter the snapping phenomenon and master the stability, the position and/or the orientation of the system, especially the distal part of the system, in order to drive it gently in the desired direction, without any undesired spring back effect. As a matter of fact, upstream the body part 6, a curvature is set up on an active area 3p, such curvature angle is higher than the tube where the system is oriented in. It aims at preventing undesired torsion of the whole elongated system so as to stabilize it during its use, for instance by a surgeon.

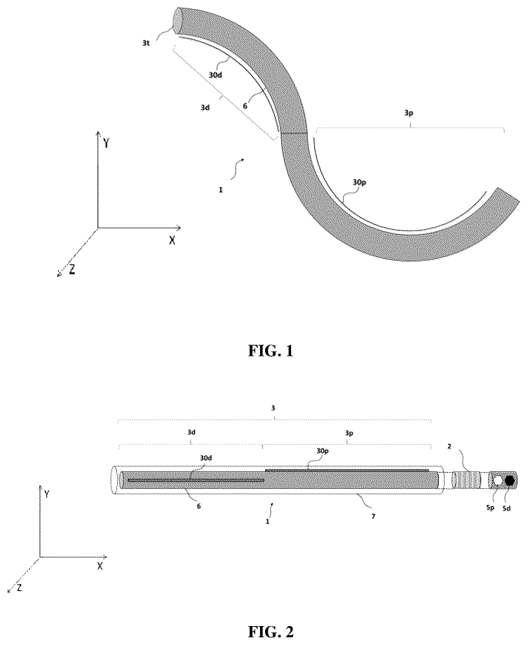

In one embodiment illustrated on FIG. 1, the system includes a body part 6, which is flexible, and presents a proximal part 2 and a distal part 3; the distal part 3 prolongs the proximal part 2; the distal part 3 is contiguous to proximal part 2, and comprises areas called active area 3p and a distal part 3d terminated by a tip 3t. According to one embodiment, said body part 6 is elongated, which means that it extends longitudinally. In one embodiment, the body part 6 is a tube. In one embodiment, the body part 6 is a catheter guide. According to one embodiment, the body part 6 is made of elastic or flexible material. In one embodiment, the body part 6 is made of polymer flexible material suitable for invasive use. In one embodiment, the body part 6 is made of a shape memory alloy. Any shape memory alloy authorized for invasive use may be used in this invention. In one embodiment, the shape memory alloy is nitinol.

In one embodiment, the system further includes at least one actuator. In one embodiment, at least one actuator 30 is fixed at at least one location of the distal part of the body part 6, so that the activation of the actuator which can be a shortening of the length of the actuator, and/or a curvature of the actuator, or a translation of the actuator towards the proximal end of the system, triggers a curvature of the distal part from the point of fixation backwards or between two points of fixation. Thus, limiting undesired torsion of the elongated system so as to stabilize it during its use, for instance by a surgeon. In one embodiment, the system includes only one actuator and this actuator is fixed to the active area 3p. In one embodiment, the system is deprived from any actuator on the functional end 3d or the tip 3t. In one embodiment, the length of the tip 3t ranges from 0 to 10 mm.

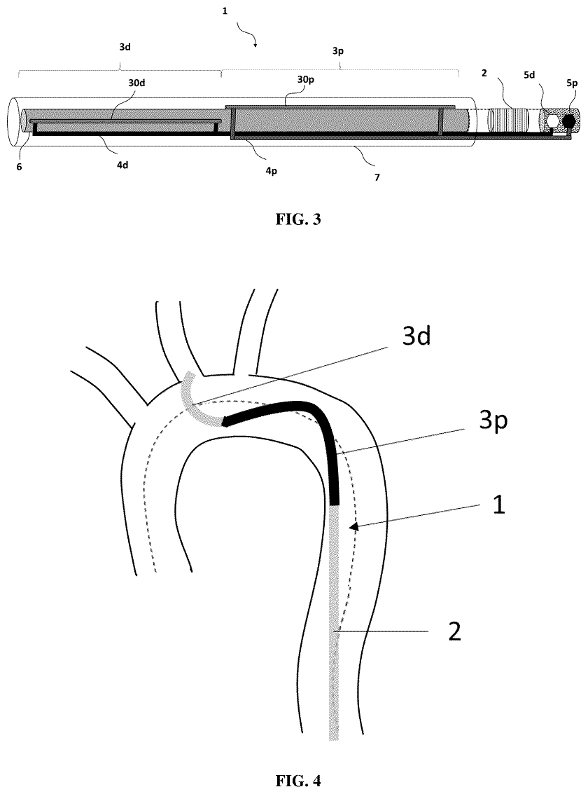

In the embodiment shown in FIG. 1 and in FIG. 2, the system further includes at least two actuators (30p, 30d); first actuator 30p and/or second actuator 30d are each independently at least partially fixed to the body part 6, preferably to the distal part 3, more preferably to the active area 3p and/or to the functional end 3d. In one embodiment, at least two actuators (30p, 30d) are not in contact one with another. The two actuators (30p, 30d) are aligned one after the other longitudinally along axis X. In FIG. 1, the two flexions/curvatures take place in the same plane XY, but it is an embodiment of the invention, of course, where the curvatures can take place in different planes. This would be the case if the actuators of FIG. 3 were actuated. This allows a better displacement within any duct or pipe or lumen.

In one embodiment, the activation of the first actuator 30p or of the second actuator 30d drives the deformation respectively of said actuators; and, as the actuators are fixed to the body part 6, the deformation of the actuators, which can be a shortening of the length of the actuator, and/or a curvature of the actuator, or a translation of the actuator towards the proximal end, results in the curvature of the body part 6. For either one or both actuators 30p and/or 30d, the actuation is able to curve the body part within an angle comprised between 0 and 180°, the angle zero being excluded. Preferably, the angle is between 5 and 180° and even more preferably between 1° and 180°.

In one embodiment shown in FIG. 2, the elongated functional system 1 further comprises a sheath 7 surrounding the body part 6.

In one embodiment illustrated on FIG. 3, the elongated functional system 1 further comprises an energy source (such as for example an electric energy source) and means for providing and/or for transmitting energy from the energy source to the first actuator 30p and/or to the second actuator 30d, said means preferably being at least one conductive wire 9. In one embodiment, a first set of conductive wires 4p is connected to the first actuator 30p. According to one embodiment, a second set of conductive wires 4d is connected to the second actuator 30d. According to one embodiment, said conductive wires (4p and 4d) are arranged to provide or to transmit an electric current or heat along their longitudinal portion to respectively the first actuator 30p and the second actuator 30d in a controlled manner. In one embodiment, the first set of conductive wires 4p is connected to the first controller 5p. According to one embodiment, the second set of conductive wires 4d is connected to the second controller 5d. In one embodiment, the first controller 5p and/or the second controller 5d allow applying an electric current to a portion of respectively the first actuator 30p and/or the second actuator 30d by means of the conductive wires 9, thereby providing the actuation of said actuators 30.

In one embodiment, the first actuator 30p and/or the second actuator 30d comprise pulling means, such as for example a pull wire or a pull cable. According to one embodiment, the pull wire or cable is fastened in the distal part of the active area 3p or of the functional end 3d and actioned at the proximal end of the proximal part 2. When a tension is applied on said pull wire in the proximal direction, the pull wire leads to bend the body part 6. The withdrawal of said tension allows the body part 6 to return to its original shape. Any pulling means able to engage a modification of the curves of the active area 3p and/or the functional end 3d may be implemented according to the invention. In an alternative embodiment, the actuators of the invention are deprived of pulling means.

In one embodiment illustrated on FIG. 4, the active area 3p is configured such that it can take the shape of any curvature of the tube where it is advanced. For example, the active area 3p takes the curve of the aortic arch. In one embodiment, the active area 3p is configured to change, when actuated, from a rest shape (for example with no curvature) to a curved shape corresponding to the curvature of the tube. In one embodiment of the invention, the system follows the curvature of the center of the tube, or is more curved than the curvature of the center of the tube, upon activation of the active area, thereby stabilizing the system against the snapping. In one embodiment, the activation of the distal end is carried out at the same time or after the activation of the active area 3p.

In one embodiment, the plane of curvature and the radius of curvature of the first curve of the active area 3p are predetermined. The deformation of the shape of the active area 3p may be designed and predefined so that it can be reproduced upon activation.

In another embodiment, shown in FIG. 1, the first actuator 30p and the second actuator 30d may be longitudinal actuators. In one embodiment, said actuators (30p, 30d) extend along the body part 6 and are fixed to body part 6. In one embodiment, the actuators (30p, 30d) arc arranged each on one side of the tube. In one embodiment, the first actuator 30p and the second actuator 30d show an angular shift with respect to one another. The angular shift ranges from 0 to 180°, preferably more than 0 to 180°. In one embodiment, the angular shift ranges from 90° to 180° In one embodiment, the angular shift is 180°. In one embodiment, the angular shift of first actuator 30p and the second actuator 30d helps leading to control the orientation of the functional end with respect to the active area, ensuring a deformation of the active area 3p and/or of the functional end 3d in predefined directions with respect to one another.

In one embodiment, the actuation and the deformation of the first actuator 30p and/or of the second actuator 30d, provides a deformation of the body part 6 and of the elongated functional system 1. In one embodiment, the first actuator 30p and/or the second actuator 30d are radially fixed to the body part 6. The first actuator 30p and the second actuator 30d respectively actuate the deformation of the active area 3p and functional end 3d of body part 6, at same of different times.

In one embodiment, the first provided curve is included in a first plane, the deformation of the active area 3p being maintained in the first plane. In one embodiment, the second curve is included in a second plane, the deformation of the functional end 3d being maintained in the second plane. When both the active area 3p and the distal functional end 3d are actuated at the same time or sequentially, the orientation of the second plane is fixed with respect to the first plane. In one embodiment, the elongated functional system 1 comprises means freezing the orientation of the second plane with respect to the first plane.

In one embodiment illustrated on FIG. 4, the functional end 3d comprises an actuator 30d and the active area 3p comprises an actuator 30p. In one embodiment, the actuator 30d extends on the body part 6 from the distal part of the first actuator 30p, optionally with an angular shift, so that the actuators are not in contact one with another. In another embodiment, the actuator 30d is spaced from the actuator 30p. According to one embodiment, said space ranges from 0 to 50 mm. According to one embodiment, said space ranges from 0 to 10 mm.

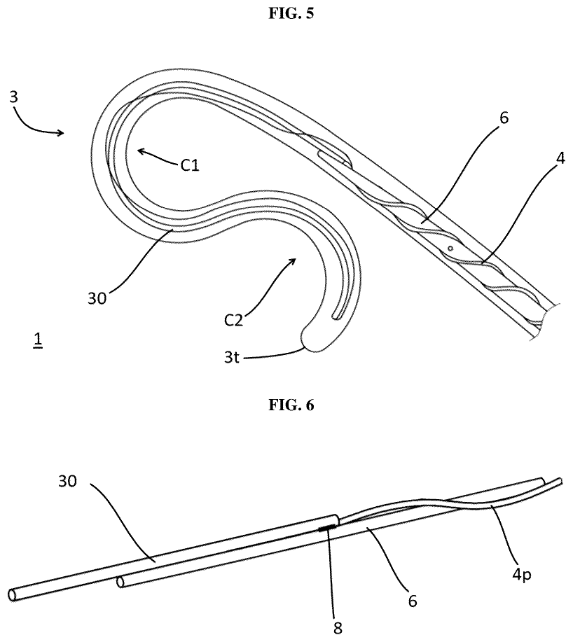

FIG. 5 illustrates one embodiment of the invention, wherein the double or multiple curvature is generated by a single actuator 30. The single actuator 30 illustrated generates a double curvature, with a first curvature C1 in a first direction and a second curvature C2 in a second direction.

In the illustrated embodiment, the first curvature C1 and the second curvature C2 are located in a same plane. In other embodiments, the first curvature C1 and the second curvature C2 are located in distinct planes, which are tilted in relation to each other.

One of the curvatures, among the first curvature C1 and the second curvature C2, is greater or equal to the curvature of the center of the tube.

The actuator 30 is controllable when it is connected to a controller 5p, which is able to deliver a variable amount of energy to the actuator. Thus, it is possible to set the amount of flexion or curvature of the active area 3p to which the actuator 30 is fixed, between the resting position and the curved position.

The multiple curvature of the actuator 30 allows to give a more complex shape to the distal tip, rather than a simple curvature. This complex shape prevents the snapping effect of the devices of the prior art, notably when the tip is approaching a junction within the tube, and it is needed to change the direction of the tip of the system 1.

In one embodiment, the first actuator 30p and/or the second actuator 30d are mechanically linked to the body part 6 with one degree of freedom in translation. According to one embodiment, the first actuator 30p and/or the second actuator 30d are mechanically linked to the body part 6 with one degree of freedom in translation along the longitudinal axis of the body part 6; which means that said mechanical linkage allows the wire to translate or retract along the longitudinal axis of the body part 6, while being retained on at least one point, preferably at least two points on the distal part 3.

FIG. 6 illustrates a partial view of a mechanical linkage between a single actuator 30 and the body 6. A welding point is located at a proximal extremity of the actuator 30 in order to set the position of the proximal extremity of the actuator 30, in relation with the body 6. Other mechanical links can be feasible, as gluing, fretting, or binding.

Setting the position of the proximal extremity only allows the remaining of the actuator 30 to move, in relation to the body 6, with at least one degree of freedom. Notably, a degree of freedom in translation allows to compensate the length modification of the actuator 30, when it is made of a shape memory alloy.

In one embodiment, the maximum radius of curvature and the direction of curvature of the first curve and the second curve are predetermined. In one embodiment, the orientation of the first plane with respect to the second plane is predetermined. In one embodiment, the predetermination is made by preparing the nitinol actuator(s) prior to assembly of the system.

According to another embodiment, the functional end 3d comprises two second actuators 30d leading to allow two predetermined orientations, radii of curvature and/or directions of curvature of the functional end 3d. According to one embodiment, the elongated functional system 1 comprises two second controllers 5d, one for each second actuator 30d. Consequently, the controllers 5p, 5d advantageously allows actuating independently each second actuators 30d, providing the functional end 3d being curved in two different pre-defined planes and two predefined radii of curvature, depending of which second actuator 30d is actuated.

In one embodiment, the distal part 3 comprises at least three actuators, each able to provide a different second curve as detailed above. The advantage is, by a modulation of the intensity of the three actuators, to control the orientation of the plane of the second curve about 360°. This embodiment advantageously allows the surgeon to correct the angle between the first plane and the second plane or the radius of curvature of the second curve during the operation if necessary e.g. if the angle between the trunk artery and the duct is different than expected.

According to one embodiment, the distal part 3 comprises at least two active areas 3p. In said embodiment, a first active area extends distally from the main body proximal part 2 and comprises a first actuator providing a first curve in a first plane when actuated. In said embodiment, a second active area is located between said first active area and the functional end and comprises a third actuator providing a third curve in a third plane when actuated. Said second active area advantageously allows providing three successive curves on the distal part 3 of the elongated functional system 1 to navigate inside complex duct system. In one embodiment, the first and the second actuator are positioned on the body part with an angular shift of more than 0°. In one embodiment, the second and the third actuator are positioned on the body part with an angular shift of more than 0°. In one embodiment, the first and the third actuators are aligned.

According to one embodiment, the distal part 3 comprises at least three active areas or a plurality of active areas as described above.

1. An elongated functional system, said system being configured to be advanced in a lumen of a pipe, a duct or a tube, said system having:
body part having a main elongated proximal part and a distal part, said distal part being a continuous extension of the proximal part, said distal part comprising one functional end terminating with a tip, and at least one active area located upstream the functional end on the distal part; and
an actuator configured to convey or transform an amount of energy to the distal part sufficient to cause a reversible multiple curvature of the active area, one curvature among the multiple curvature being greater or equal to the curvature of the center of the tube, this tube curvature being supposed to be non zero, at the location of the active area, thereby preventing snapping effect of the whole system; the actuator being connectable to a source of energy.

2. The elongated functional system according to claim 1, wherein the actuator and, thereby, the active area, are distant from the tip, a distance between the distal end of the tip and the distal end of being of 0.1 to 5 times a length of the actuator the actuator.

3. The elongated functional system according to claim 1, wherein the main elongated proximal part and/or the distal part are straight and flexible.

4. The elongated functional system according to claim 1, wherein the functional end is straight or curved by design, and flexible.

5. The elongated functional system according to claim 1, wherein the actuator is made of, or comprise, at least one shape memory alloy or at least one polymer or at least one metal or at least one piezo-electric material.

6. The elongated functional system according to claim 1, wherein the actuator is made of shape memory alloy.

7. The elongated functional system according to claim 1, wherein actuator contracts or curvates when stimulated by a source of energy, thereby respectively: causing a flexion or curvature of the active area to which it is fixed, and/or activating the functional end.

8. The elongated functional system according to claim 1, wherein the functional end is a steerable end.

9. The elongated functional system according to claim 1, wherein the distal part comprises at least one second actuator.

10. The elongated functional system according to claim 9, wherein the actuator and/or the at least one second actuator, when activated, trigger a simple, a double, or a multiple curvature and selected in the group consisting of an S-shape, a C-shape, a U-shape, an L-shape, a J-shape and a G-shape of whole or part of the distal part.

11. The elongated functional system according to claim 1, further comprising an external control unit located at the proximal end of part, said unit comprising at least one controller device configured to actuate independently each actuator.

12. The elongated functional system according to claim 1, further comprising a source of energy and means for providing energy to the actuator connected to said source of energy.

13. The elongated functional system according to claim 1, which is a catheter guide.

14. A method for stabilizing an elongated functional system advanced in a lumen of a of a pipe, a duct or a tube having at least one curve, wherein the elongated functional system has:
a body part having a main proximal part and a distal part, said distal part being a continuous extension of the proximal part, said distal part comprising one functional end terminating with a tip, and at least one active area located upstream the functional end on the distal part; and
an actuator configured to convey or transform an amount of energy to the distal part sufficient to cause a reversible curvature of the active area, greater or equal to the curvature of the center of the tube, at the location of the active area, thereby preventing undesired rotation of the whole system; the actuator being connectable to a source of energy,
the method comprising the steps of:
advancing the system in a curve of the tube;
stabilizing the system by actuating the active area so that it reaches a multiple curvature, one curvature among the multiple curvature being equal or greater than that of the curve of the tube.
